(19) United States
(12) Patent Application Publication
(10) Pub. No.: US 2009/0280175 A1
(43) Pub. Date: Nov. 12, 2009

(54) Multilayer Proton Pump Inhibitor Tablets

(76) Inventors: Ishwar Chauhan, Voorhees, NJ (US); Siva Rama Krishna Nutalapati, Princeton, NJ (US)

Correspondence Address: Licata & Tyrrell P.C., 66 E. Main Street, Marlton, NJ, 08053, US

(21) Appl. No.: 12/436,823

(22) Filed: May 7, 2009

Related U.S. Application Data
(60) Provisional application No. 61/051,745, filed on May 9, 2008.

(51) Int. Cl. A61K 9/24; A61K 31/4439

(52) U.S. Cl. 424/472; 514/338

(57) ABSTRACT

Multilayer tablets of a proton pump inhibitor essentially bioequivalent in terms of plasma Cₘₐₓ and AUC to capsules and/or tablets consisting of multiple unit pellets of the proton pump inhibitor are provided. Also provided are methods for production of these multilayer tablets and methods for their use in treating dyspepsia, peptic ulcer disease, gastroesophageal reflux disease and Zollinger-Ellison syndrome.

This patent application claims the benefit of priority from U.S. Provisional Application Ser. No. 61/051,745 filed May 9, 2008, which is herein incorporated by reference in its entirety.

### FIELD OF THE INVENTION

The present invention relates to enteric coated multilayer tablets of a proton pump inhibitor, which are bioequivalent in terms of plasma Cₘₐₓ and AUC to capsules and/or tablets comprising multiple unit pellet systems of the proton pump inhibitor.

### BACKGROUND OF THE INVENTION

Proton pump inhibitors (PPIs) such as, but not limited to, Omeprazole, Esomeprazole, Lansoprazole, Pantoprazole and Rabeprazole sodium and/or salts thereof are used in the treatment of dyspepsia, peptic ulcer disease, gastroesophageal reflux disease and Zollinger-Ellison syndrome. PPIs inhibit the gastric enzyme H⁺, K⁺-ATPase (the proton pump) which catalyzes the exchange of H⁺ and K⁺. PPIs are effective in the inhibition of both basal acid secretion and stimulated acid secretion. This inhibition is dose-dependent and daily oral doses of, for example, Omeprazole at 20 mg and higher exhibit consistent and effective acid control.

Omeprazole was first marketed by AstraZeneca under the trade names LOSEC and PRILOSEC. Esomeprazole, the S-enantiomer of omeprazole was also developed and is marketed by AstraZeneca under the trade names LOSEC and PRILOSEC. The magnesium salt of esomeprazole is marketed under the tradename NEXIUM.

Lansoprazole has been marketed for many years and is one of several PPIs available. Lansoprazole is the active ingredient in PREVACID.

Pantoprazole was developed by Altana and is currently marketed under the brand name PROTONIX by Wyeth-Ayerst Laboratories, SOMAC by Pfizer, ASTROPAN by Astron Lifesciences, PANTOR by Bosnalijek, and PANTOLOC by Solvay Pharma, and as PROTIUM in the United Kingdom and as Topzole in South Africa.

Rabeprazole sodium is the active ingredient in ACIPHEX, manufactured by Eisai Inc. and PriCara Division of Ortho-McNeil-Janssen Pharmaceuticals, Inc.

Most proton pump inhibitors are available as tablets and capsules (containing the proton pump inhibitor or salt thereof) in strengths of 10 mg, 20 mg, and in some markets 40 mg; and as a powder for intravenous injection. Most oral proton pump inhibitor preparations are enteric-coated, due to the rapid degradation of the drug in the acidic conditions of the stomach. Enteric protection is most commonly achieved by formulating enteric-coated granules within capsules, enteric-coated tablets, and enteric-coated multiple-unit pellet system commonly referred to as MUPS compressed into tablets.

For example, Omeprazole magnesium tablets manufactured by AstraZeneca (Prilosec OTC) are formulated as a “multiple unit pellet system” (MUPS). Essentially, the tablet consists of extremely small enteric-coated granules (pellets) of Omeprazole magnesium compressed into tablets using acceptable pharmaceutical excipients.

In June 2004 the FDA approved an immediate release preparation of Omeprazole/Sodium Bicarbonate that does not require an enteric coating. In this preparation Sodium Bicarbonate acts as a buffering agent to protect Omeprazole from gastric degradation. This combination preparation is marketed in the United States by Santarus under the trade name ZEGERID. ZEGERID is marketed as capsules, chewable tablets, and a powder for oral suspension. ZEGERID is most useful for those patients who suffer from nocturnal acid breakthrough (NAB) or those patients who desire immediate relief.

Information from clinical trials in patients with duodenal ulcers in remission indicates that enteric coated proton pump inhibitors such as Omeprazole magnesium 20 mg tablets (as a single unit formulation) demonstrate the same inhibition of stimulated acid secretion and similar effect on 24-hour intragastric pH as Omeprazole magnesium 20 mg capsules (as a multiple unit formulation). The mean decrease in peak acid output after pentagastrin stimulation was approximately 70%, after 5 days of dosing with enteric coated Omeprazole magnesium 20 mg tablets once daily. However, enteric coated Omeprazole magnesium 20 mg tablets (as a single unit formulation) and Omeprazole magnesium 20 mg capsules (as a multiple unit formulation) are not bioequivalent in terms of plasma AUC, Cₘₐₓ and tₘₐₓ. The enteric coated Omeprazole magnesium 20 mg tablets demonstrate, after repeated dosing, increased plasma Omeprazole AUC (18%) and maximum concentration (41%) in comparison to Omeprazole magnesium 20 mg given as capsules (as a multiple unit formulation). The Omeprazole magnesium 20 mg capsule (as a multiple unit formulation) is usually emptied gradually from the stomach into the intestine. In contrast to the capsule, the enteric coated tablet (as a single unit formulation) enters the intestine and dissolves as one unit. Consequently, the absorption and first pass metabolism of the tablet take place only during a very limited period. This may be one of the reasons for the difference observed in the pharmacokinetic variables of the two formulations.

Such differences in the pharmacokinetic parameters make substituting the multiple unit pellet formulation of proton pump inhibitors with a single unit tablet formulation extremely difficult.

### SUMMARY OF THE INVENTION

An aspect of the present invention relates to an enteric coated multilayer tablet of a proton pump inhibitor which is essentially bioequivalent in terms of plasma Cₘₐₓ and AUC to capsules and tablets consisting of multiple unit pellets of the proton pump inhibitor. Preferred in the multilayer tablets of the present invention is that the proton pump inhibitor not comprise Omeprazole or a salt thereof. Examples of proton pump inhibitors preferred for use in the present invention include, but are not limited to, Esomeprazole, Lansoprazole, Pantoprazole and Rabeprazole sodium, or a salt thereof.

In one embodiment, the multilayer tablet comprises a core region with one or more immediate release proton pump inhibitor containing layers or portions and one or more extended release proton pump inhibitor containing layers or portions.

In another embodiment, the multilayer tablet comprises a core region containing a proton pump inhibitor; a polymer layer coating the core region which provides for slow release of the proton pump inhibitor from the core region, and a proton pump inhibitor containing top layer coating the polymer layer which rapidly releases the proton pump inhibitor in the layer upon contact of the tablet with fluid.

Upon reaching the small intestine, the multilayer tablets of the present invention release the proton pump inhibitor at a rate which achieves an acceptable plasma Cₘₐₓ and AUC of the proton pump inhibitor as compared to capsules and/or tablets consisting of multiple unit pellets of the proton pump inhibitor.

Another aspect of the present invention relates to methods for formulating a proton pump inhibitor as a multilayer tablet which is essentially bioequivalent in terms of plasma Cₘₐₓ and AUC to capsules and/or consisting of multiple unit pellets of the proton pump inhibitor.

In one embodiment of this method, the multilayer tablet comprising a core region with one or more immediate release portions or layers of a proton pump inhibitor and one or more extended release portions or layers of the proton pump inhibitor are compressed together into a tablet. This tablet is then coated with an enteric polymer to protect it from gastric environment. In some embodiments, the tablet is coated with a subcoating prior to coating with the enteric polymer.

In another embodiment, the multilayer tablet comprising a core region containing a proton pump inhibitor is compressed into a tablet. A polymer layer coating which provides for slow release of the proton pump inhibitor from the core region is then applied to the tablet. A top layer containing the proton pump inhibitor, which rapidly releases the proton pump inhibitor in the layer upon of the tablet coming into contact with a fluid, is then applied as a coating to the polymer layer.

Another aspect of the present invention relates to a method for treating dyspepsia, peptic ulcer disease, gastroesophageal reflux disease and Zollinger-Ellison syndrome which comprises administering to a patient a multilayer tablet of a proton pump inhibitor comprising either a core region with one or more immediate release proton pump inhibitor containing layers or portions and one or more extended release proton pump inhibitor containing layers or portions or a core region containing the proton pump inhibitor, a polymer layer coating the core region which provides for slow release of the proton pump inhibitor from the core region, and a proton pump inhibitor containing top layer coating the polymer layer which rapidly releases the proton pump inhibitor in the layer upon contact of the tablet with fluid, wherein the multilayer tablet exhibits an essentially bioequivalent plasma Cₘₐₓ and AUC to capsules and/or tablets consisting of multiple unit pellets of the proton pump inhibitor.

### DETAILED DESCRIPTION OF THE INVENTION

The present invention provides multilayer tablets of a proton pump inhibitor which exhibit acceptable plasma Cₘₐₓ and AUC as compared to capsules and/or tablets consisting of multiple unit pellets of the proton pump inhibitor.

By “proton pump inhibitor” or “PPI” as used herein, it is meant a drug whose main action is pronounced and long-lasting reduction of gastric acid production. These drugs are among the most widely-selling drugs in the world as a result of their outstanding efficacy and safety. Structurally, the vast majority of PPIs are benzimidazole derivatives; however, imidazopyridine derivatives may provide a more effective PPI. Further, by proton pump inhibitor or PPI as used herein it is meant to be inclusive of pharmaceutically acceptable salts of the PPI. Examples of PPIs include, but are not limited to, Omeprazole, Esomeprazole, Lansoprazole, Pantoprazole and Rabeprazole sodium, and salt thereof. Preferred in the multilayer tablets of the present invention is that the PPI not comprise Omeprazole or a salt thereof. Examples of preferred PPIs for use in the multilayer tablets of the present invention include, but are not limited to, Esomeprazole, Lansoprazole, Pantoprazole and Rabeprazole sodium and salts thereof.

Either crystalline forms or amorphous forms of the PPI can be used.

By “acceptable” plasma Cₘₐₓ and AUC, as used herein, it is meant that the multilayer tablets of the present invention exhibit plasma Cₘₐₓ and AUC within 80 to 120% of the plasma Cₘₐₓ and AUC of the FDA approved capsules and/or tablets consisting of multiple unit pellets of a selected PPI. The multilayer tablets of the present invention thus provide for controlled release of a PPI over a period of time compatible with the desired time needed and prevent the high plasma concentrations that are otherwise observed with immediate release dosage formulations.

In one embodiment of the present invention the PPI containing tablets are formulated to release the PPI over a predetermined time period by preparing a core region of the tablet with multiple layers or portions containing the PPI with different release patterns. In simplest form, the tablet of the present invention comprises a bilayer core region wherein the first layer or portion of the core region is an immediate release layer or portion and the second layer or portion of the core region is an extended release layer or portion. As will be understood by the skilled artisan upon reading this disclosure, tablets of the present invention may comprise additional immediate release layers or portions and/or additional extended release layers or portions.

The immediate release layer or layers or portion or portions is that part of the core region of the tablet with a dissolution profile from 0 to 20 minutes in a suitable in vitro dissolution test. A suitable exemplary dissolution test is set forth in Example 1 herein. In this exemplary test, dissolution is carried out in 900 mL of phosphate buffer (pH 6.8) at temperature of 37.0° C.±0.5° C. using apparatus I (basket) rotating at a speed of 100 rpm. However, as will be understood by the skilled artisan upon reading this disclosure, variations on this test as well as the apparatus and conditions well known to those skilled in the art can be used. In one embodiment of the present invention, 80% or more of the PPI in the immediate release layer or layers or portion or portions of the core region is dissolved in 20 minutes, and more preferably in 15 minutes.

The extended release layer or layers or portion or portions of the core region of a multilayer tablet of the present invention is that part of the core region of the tablet with a dissolution profile which is after 15 to 20 minutes, measured in a suitable in vitro dissolution test, such as described herein in Example 1. In one embodiment of the present invention, the complete dissolution time of the PPI in the extended release layer or layers or portion or portions of the core region is within 120 minutes, and preferably within 90 minutes, of administration.

Tablets of the present invention can be prepared by methods and contain vehicles which are well-known in the art. Generally recognized compendiums of such methods and ingredients include Remington: The Science and Practice of Pharmacy, Alfonso R. Gennaro, editor, 20th ed. Lippincott Williams & Wilkins: Philadelphia, Pa., 2000 and Sheth et al. Compressed Tablets, in Pharmaceutical Dosage Forms: Tablets, Vol 1. edited by H. A. Lieberman and L. Lachman, Dekker N.Y. (1980).

In one embodiment, the immediate release layer or layers or portion or portions of the core region of a multilayer tablet of the present invention is prepared by direct compression of a mixture of the PPI with a suitable carrier or excipient, such as carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, sodium carboxymethylcellulose, or microcrystalline cellulose; gums including arabic and tragacanth; proteins such as gelatin and collagen; inorganics, such as kaolin, calcium carbonate, dicalcium phosphate, sodium chloride; magnesium carbonate; magnesium oxide; and other agents such as acacia and alginic acid.

Agents that facilitate disintegration and/or solubilization can also be added, such as the cross-linked polyvinyl pyrrolidone, sodium starch glycolate, Croscarmellose Sodium, alginic acid, or a salt thereof, such as sodium alginate, microcrystalline cellulose and corn starch.

Tablet binders that can be used include acacia, methylcellulose, sodium carboxymethylcellulose, polyvinylpyrrolidone (povidone), hydroxypropyl cellulose, hydroxypropyl methylcellulose, sucrose, starch and ethylcellulose.

Lubricants that can be used include magnesium stearates, stearic acid, sodium Stearyl fumerate, talc, waxes, oils, silicon dioxide and colloidal silica.

Fillers, agents that facilitate disintegration and/or solubilization, tablet binders and lubricants, including the aforementioned, can be used singly or in combination.

The immediate release layer or layers of the tablets are then formulated, for example, by preparing a powder mixture by dry blending or granulating or slugging, adding a lubricant and disintegrant and pressing into tablets layers.

The extended release layer or layers or portion or portions of the core region of the tablet can be prepared by incorporating matrix-forming excipients and/or non-matrix forming excipients into the above-described formulation for the immediate release layer or portion, and either completely omitting or reducing the amount of disintegrants.

Examples of matrix-forming excipients include, but are not limited to hydrophilic polymers such as hydroxypropylmethylcellulose, hydroxymethylcellulose, hydroxypropylcellulose and hydroxyethylcellulose, and which swell in contact with aqueous liquids, and control release of the drug by diffusion through the swollen polymer network, and are incorporated at a level between 5 and 50% by weight with respect to that of the extended release portion of the tablet.

Examples of non-matrix-forming excipients include, but are not limited to, waxes such as carnauba wax, bees wax stearic acid and gums such as acacia and are incorporated at a level between 5 and 50% by weight with respect to that of the extended release portion of the tablet.

The extended release layer or layers of the tablets are then formulated, for example, by preparing the powder mixture by dry blending or granulating or slugging, adding a lubricant and matrix-forming excipients and/or non-matrix forming excipients and pressing into tablet layers.

One or more of the immediate release layers and one or more of the extended release layers are then compressed together to form a single core region for a multilayer tablet of the present invention. In this embodiment, 10% to 90%, preferably 15% to 85%, more preferably 20% to 80%, of the PPI is in the immediate release layer or layers and 90% to 10%, preferably 85% to 15%, more preferably 80% to 20%, of the PPI is in the extended release layer or layers.

Alternatively the core region of the tablets may be prepared by granulation with water of a mixture of the drug or salts thereof with suitable diluents, disintegrant and binding polymer; calibration and drying of the granulate addition of a lubricant, followed by compression on a tableting machine.

In one embodiment, the core region of the tablets is then coated with an enteric polymer. Examples of enteric polymers include, but are not limited to, polymers such as methacrylic acid-ethyl acrylate copolymer (1:1), ethacrylic acid-methyl methacrylate copolymer (1:1), methacrylic acid-methyl methacrylate copolymer (1:2), polyvinyl acetate phthalate (PVAP), hydroxypropyl methylcellulose acetate succinate (HPMCAS) and cellulose acetate phthalate (CAP). Additionally, dyestuffs or pigments can be added to the enteric polymer coating for product identification or to characterize the quantity of active compound, i.e., dosage.

In some embodiments, prior to applying the enteric polymer coating, the core region is coated with a subcoating and then coated with the enteric polymer coating to avoid drug interactions with the enteric polymer. Examples of polymers used for subcoating include, but are not limited to polymers such as polyvinyl pyrrolidine, hydroxymethyl cellulose, hydroxypropylmethylcellulose, and hydroxypropylcellulose.

In an alternative embodiment, the core region of the tablets is coated with an extended release polymer. Examples of extended release polymers include, but are not limited to polymers such as ethyl cellulose, hydroxypropyl methyl cellulose, ammonio methacrylate copolymer (Type A), ammonio methacrylate copolymer (Type B) and ethyl acrylate methyl methacrylate copolymer dispersion. These polymers can be used alone or in combination with other extended release, immediate release or enteric polymers.

In some embodiments, prior to applying the extended release polymer coating, the core region is coated with a subcoating and then coated with the extended release polymer coating.

In an alternative embodiment, the multilayer tablet of the present invention comprises a core region comprising a PPI, a polymer layer coating the core region which provides for slow release of the PPI from the core region, and a PPI containing top layer applied over the polymer coating which rapidly releases the PPI in the layer upon of the tablet coming into contact with a fluid.

In this embodiment the core region is formulated, for example, by preparing a powder mixture by dry blending or granulating or slugging, adding a lubricant and pressing into tablets. The core region of the tablets is then coated with an extended release polymer. Examples of extended release polymers include, but are not limited to polymers such as ethyl cellulose, hydroxypropyl methyl cellulose, ammonio methacrylate copolymer (Type A), ammonio methacrylate copolymer (Type B) and ethyl acrylate methyl methacrylate copolymer dispersion. Additionally, these polymers can be used alone or in combination with other extended release or enteric polymers.

The immediate release layer comprising a PPI and the one or more excipients is deposited onto the core in the form of a solution or suspension comprising an aqueous buffer solvent or nonaqueous solvent such as, for example, methyl alcohol, ethyl alcohol or isopropyl alcohol.

In one embodiment, the immediate-release portion is in the form of a coating substantially surrounding the coated core applied, for example, using spray coating, compression coating, or other suitable technique. This coating comprises a PPI and one or more excipients such as a disintegrant (e.g., crospovidone, croscarmellose sodium, pregelatinized starch), a binder (e.g., povidone, Hydroxypropyl methyl cellulose), a plasticizer (e.g., polyethylene glycol, Triethyl citrate), a lubricant (e.g., magnesium stearate, talc), a filler (e.g., microcrystalline cellulose, colloidal silicon dioxide), solubilizing and/or wetting agents (e.g., polysorbate 80), and combinations comprising one or more of the foregoing excipients.

These tablets are then coated with an enteric polymer. Examples of enteric polymers include, but are not limited to polymers such as methacrylic acid-ethyl acrylate copolymer (1:1), methacrylic acid-methyl methacrylate copolymer (1:1), methacrylic acid-methyl methacrylate copolymer (1:2), polyvinyl acetate phthalate (PVAP), hydroxypropyl methylcellulose acetate succinate (HPMCAS) and cellulose acetate phthalate (CAP). Additionally, dyestuffs or pigments can be added to the enteric polymer coating for product identification or to characterize the quantity of active compound, i.e., dosage.

In some embodiments, prior to applying the enteric polymer coating, the tablets are coated with a subcoating and then coated with the enteric polymer coating to avoid drug interactions with the enteric polymer. Examples of polymers used for subcoating include, but are not limited to, polymers such as poly vinyl pyrrolidine, hydroxymethyl cellulose, hydroxypropylmethylcellulose, and hydroxypropylcellulose.

In an alternative embodiment, the tablets are coated with an extended release polymer. Examples of extended release polymers include, but are not limited to polymers such as ethyl cellulose, hydroxypropyl methyl cellulose, ammonio methacrylate copolymer (Type A), ammonio methacrylate copolymer (Type B) and ethyl acrylate methyl methacrylate copolymer dispersion. Additionally, these polymers can be used alone or in combination with other extended release or enteric polymers.

In some embodiments, prior to applying the extended release polymer coating, the core region is coated with a subcoating and then coated with the extended release polymer coating.

Dosage forms where the immediate release entity and the extended release entity are administered simultaneously but separately are also encompassed by the present invention.

The following nonlimiting examples are provided to further illustrate the present invention. While the PPI in these examples is Omeprazole, as will be understood by the skilled artisan upon reading this disclosure, alternative PPIs can be routinely substituted in the described exemplary formulations.

### EXAMPLES

### Example 1

### Preparation of Enteric-Coated Multilayer Tablet with 20% Omeprazole in Immediate Release Layer and 80% Omeprazole in Extended Release Layer

The immediate release layer or portion contained Omeprazole magnesium (4.49 mg/tablet), microcrystalline cellulose (38.51 mg/tablet), lactose anhydrous (50.00 mg/tablet), hydroxypropyl cellulose (3.00 mg/tablet), croscarmellose sodium (3.00 mg/tablet), and magnesium stearate (1.00 mg/tablet).

The extended release layer or portion contained Omeprazole magnesium (17.96 mg/tablet), microcrystalline cellulose (100.04 mg/tablet), lactose anhydrous (50.00 mg/tablet), hydroxypropyl cellulose (30.00 mg/tablet), and magnesium stearate (2.00 mg/tablet).

The subcoating contained Opadry II Clear (10 mg/tablet) and purified water which was removed during processing.

The enteric coating contained Eudragit L30D55 (24.32 mg/tablet), triethyl citrate (2.66 mg/tablet), talc (14.62 mg/tablet) and purified water which was removed during processing.

The immediate release layer and the extended release layer were prepared as follows:

Omeprazole magnesium was dry blended with all the ingredients except magnesium stearate for five minutes in a blender. Magnesium stearate was screened and then added to the blender. The mixture was then blended for another 2 minutes.

The layers were then compressed into a bi-layer tablet using a bi-layer tablet press.

The subcoating was prepared by dissolving Opadry II Clear in purified water and sprayed as a coating solution onto the bilayer tablet bed in a coating pan.

The enteric coating was prepared by mixing Eudragit L30D55 and triethyl citrate in a container using a mixer. In a separate container purified water was mixed with talc using mixer until the talc is evenly dispersed in the water. The talc suspension was then added to the Eudragit dispersion and mixed for 15 minutes. The resulting dispersion was mixed during the entire coating process. Using the coating pan, the Eudragit/Talc dispersion was sprayed onto the sub-coated tablets until the required weight gain of 41.6 mg/tablet was achieved.

### Example 2

### Preparation of Enteric-Coated Multilayer Tablet with 70% Omeprazole in Immediate Release Layer and 30% Omeprazole in Extended Release Layer

The immediate release layer or portion contained Omeprazole magnesium (15.72 mg/tablet), microcrystalline cellulose (37.28 mg/tablet), lactose anhydrous (40.00 mg/tablet), hydroxypropyl cellulose (3.00 mg/tablet), croscarmellose sodium (3.00 mg/tablet), and magnesium stearate (1.00 mg/tablet).

The extended release layer or portion contained Omeprazole magnesium (6.73 mg/tablet), microcrystalline cellulose (101.27 mg/tablet), lactose anhydrous (60.00 mg/tablet), hydroxypropyl cellulose (30.00 mg/tablet), and magnesium stearate (2.00 mg/tablet).

The subcoating contained Opadry II Clear (10 mg/tablet) and purified water which was removed during processing.

The enteric coating contained Eudragit L30D55 (24.32 mg/tablet), triethyl citrate (2.66 mg/tablet), talc (14.62 mg/tablet) and purified water which was removed during processing.

Tablets were prepared as described in Example 1.

### Example 3

### Preparation of Multilayer Tablet with 20% Omeprazole in Immediate Release Layer and 80% Omeprazole in Extended Release Layer

The immediate release layer or portion contained Omeprazole magnesium (4.49 mg/tablet), microcrystalline cellulose (18.00 mg/tablet), lactose anhydrous (70.51 mg/tablet), hydroxypropyl cellulose (3.00 mg/tablet), croscarmellose sodium (3.00 mg/tablet), and magnesium stearate (1.00 mg/tablet).

The extended release layer or portion contained Omeprazole magnesium (17.96 mg/tablet), microcrystalline cellulose (25.00 mg/tablet), lactose anhydrous (130.04 mg/tablet), hydroxypropyl cellulose (25.00 mg/tablet), and magnesium stearate (2.00 mg/tablet).

Omeprazole magnesium was dry blended with all the ingredients except magnesium stearate for five minutes in a blender. Magnesium stearate was screened and then added to the blender. The mixture was then blended for another 2 minutes.

The layers were then compressed into a bi-layer tablet using a bi-layer tablet press.

### Example 4

### Preparation of Multilayer Tablet with 50% Omeprazole in Immediate Release Layer and 50% Omeprazole in Extended Release Layer

The immediate release layer or portion contained Omeprazole magnesium (11.35 mg/tablet), microcrystalline cellulose (31.65 mg/tablet), lactose anhydrous (50.00 mg/tablet), hydroxypropyl cellulose (3.00 mg/tablet), croscarmellose sodium (3.00 mg/tablet), and magnesium stearate (1.00 mg/tablet).

The extended release layer or portion contained Omeprazole magnesium (11.35 mg/tablet), microcrystalline cellulose (86.65 mg/tablet), lactose anhydrous (50.00 mg/tablet), hydroxypropyl cellulose (50.00 mg/tablet), and magnesium stearate (2.00 mg/tablet).

Omeprazole magnesium was dry blended with all the ingredients except magnesium stearate for five minutes in a blender. Magnesium stearate was screened and then added to the blender. The mixture was then blended for another 2 minutes.

The layers were then compressed into a bi-layer tablet using a bi-layer tablet press.

### Example 5

### Preparation of Compression Coated Multilayer Tablet with 50% Omeprazole in Immediate Release Layer and 50% Omeprazole in Extended Release Layer

The extended release layer core contained Omeprazole magnesium (11.35 mg/tablet), microcrystalline cellulose (32.00 mg/tablet), lactose anhydrous (95.15 mg/tablet), hydroxypropyl cellulose (5.00 mg/tablet), croscarmellose sodium (5.00 mg/tablet) and magnesium stearate (1.50 mg/tablet).

The extended release layer coating contained ethyl cellulose (6.00 mg/tablet), hydroxypropyl methyl cellulose (1.50 mg/tablet), polyethylene glycol (0.75) and ethyl alcohol which is removed during processing.

The immediate release layer compression coating contained Omeprazole magnesium (11.35 mg/tablet), microcrystalline cellulose (32.00 mg/tablet), lactose anhydrous (95.15 mg/tablet), hydroxypropyl cellulose (5.00 mg/tablet), croscarmellose sodium (5.00 mg/tablet), and magnesium stearate (1.50 mg/tablet).

The subcoating contained Opadry II Clear (10 mg/tablet) and purified water which was removed during processing.

The enteric coating contained Eudragit L30D55 (24.32 mg/tablet), triethyl citrate (2.66 mg/tablet), talc (14.62 mg/tablet) and purified water which was removed during processing.

The extended release layer core was prepared by dry blending Omeprazole magnesium with all the ingredients except magnesium stearate for five minutes in a blender.

Magnesium stearate was screened and then added to the blender. The mixture was then blended for another 2 minutes and compressed into a tablet using a tablet press.

The extended release layer coating was prepared by dissolving ethyl cellulose and hydroxypropyl methyl cellulose in ethyl alcohol. Polyethylene glycol was then added and the solution was mixed. The solution was then sprayed on the extended release layer core tablet.

The immediate release layer compression coating was prepared by dry blending Omeprazole magnesium with all the ingredients except magnesium stearate for five minutes in a blender. Magnesium stearate was screened and then added to the blender. The mixture was then blended for another 2 minutes and compress coated onto the coated extended release cores using a multilayer tablet press.

The subcoating was prepared by dissolving Opadry II Clear in purified water and sprayed as a coating solution onto the bilayer tablet bed in a coating pan.

The enteric coating was prepared by mixing Eudragit L30D55 and triethyl citrate in a container using a mixer. In a separate container purified water was mixed with talc using mixer until the talc is evenly dispersed in the water. The talc suspension was then added to the Eudragit dispersion and mixed for 15 minutes. The resulting dispersion was mixed during the entire coating process. Using the coating pan, the Eudragit/Talc dispersion was sprayed onto the sub-coated tablets until the required weight gain of 41.6 mg/tablet was achieved.

### Example 6

### Preparation of Spray Coated Multilayer Tablet with 50% Omeprazole in Immediate Release Layer and 50% Omeprazole in Extended Release Layer

The extended release layer core contained Omeprazole magnesium (11.35 mg/tablet), microcrystalline cellulose (32.00 mg/tablet), lactose anhydrous (95.15 mg/tablet), hydroxypropyl cellulose (5.00 mg/tablet), croscarmellose sodium (5.00 mg/tablet) and magnesium stearate (1.50 mg/tablet).

The extended release layer coating contained ethyl cellulose (6.00 mg/tablet), hydroxypropyl methyl cellulose (1.50 mg/tablet), polyethylene glycol (0.75) and ethyl alcohol which is removed during processing.

The immediate release layer coating contained Omeprazole magnesium (11.35 mg/tablet), lactose anhydrous (79.65 mg/tablet), hydroxypropyl cellulose (5.00 mg/tablet) and croscarmellose sodium (5.00 mg/tablet).

The subcoating contained Opadry II Clear (10 mg/tablet) and purified water which was removed during processing.

The enteric coating contained Eudragit L30D55 (24.32 mg/tablet), triethyl citrate (2.66 mg/tablet), talc (14.62 mg/tablet) and purified water which was removed during processing.

The extended release layer core was prepared by dry blending Omeprazole magnesium with all the ingredients except magnesium stearate for five minutes in a blender. Magnesium stearate was screened and then added to the blender. The mixture was then blended for another 2 minutes and compressed into a tablet using a tablet press.

The extended release layer coating was prepared by dissolving ethyl cellulose and hydroxypropyl methyl cellulose in ethyl alcohol. Polyethylene glycol was then added and the solution was mixed. The solution was then sprayed on the extended release layer core tablet.

The immediate release layer coating was prepared by dispersing and/or dissolving the ingredients in an aqueous buffer (pH 9.0). The mixture was then sprayed onto the extended release cores in a suitable coating pan.

The subcoating was prepared by dissolving Opadry II Clear in purified water and sprayed as a coating solution onto the bilayer tablet bed in a coating pan.

The enteric coating was prepared by mixing Eudragit L30D55 and triethyl citrate in a container using a mixer. In a separate container purified water was mixed with talc using mixer until the talc is evenly dispersed in the water. The talc suspension was then added to the Eudragit dispersion and mixed for 15 minutes. The resulting dispersion was mixed during the entire coating process. Using the coating pan, the Eudragit/Talc dispersion was sprayed onto the sub-coated tablets until the required weight gain of 41.6 mg/tablet was achieved.

### Example 7

### Dissolution Profiles of Various Tablets

Dissolution profiles for Omeprazole magnesium core tablets, 20 mg, produced in accordance with Examples 1 through 4 were assessed in phosphate buffer, pH 6.8, using baskets rotating at 100 rpm. Results are shown in the Table 1.

TABLE 1

| Time | | | | |
|---|---|---|---|---|
| (min) | Ex. 1 | Ex. 2 | Ex. 3 | Ex. 4 |
| 0 | 0 | 0 | 0 | 0 |
| 5 | 20 | 64 | 24 | 41 |
| 10 | 40 | 71 | 34 | 47 |
| 15 | 60 | 75 | 41 | 49 |
| 20 | 74 | 77 | 50 | 52 |
| 25 | 80 | 79 | 64 | 55 |
| 30 | 85 | 81 | 82 | 57 |
| 45 | 90 | 86 | 95 | 61 |
| 60 | 92 | 90 | 98 | 66 |

Dissolution profiles for Omeprazole magnesium enteric coated tablets, 20 mg, produced in accordance with Examples 1 and 2 were assessed in phosphate buffer, pH 6.8, using baskets rotating at 100 rpm. Results are shown in the Table 2.

TABLE 2

| Time | | | | |
|---|---|---|---|---|
| (min) | Ex. 1 | Ex. 2 | Ex. 3 | Ex. 3 |
| 0 | 0 | 0 | 0 | 0 |
| 5 | 0 | 0 | 0 | 0 |
| 10 | 0 | 0 | 0 | 0 |
| 15 | 0 | 2 | 0 | 0 |
| 20 | 10 | 18 | 8 | 15 |
| 25 | 15 | 54 | 13 | 48 |
| 30 | 28 | 65 | 24 | 59 |
| 45 | 46 | 78 | 39 | 70 |
| 60 | 65 | 82 | 60 | 77 |

What is claimed is:

1. A multilayer tablet comprising a core region with one or more immediate release layers or portions containing a proton pump inhibitor and one or more extended release layers or portions of the proton pump inhibitor, where the ratio of proton pump inhibitor in the one or more immediate release layers or portions to that in one or more extended release layers or portions ranges from 10:90 to 90:10.

2. A multilayer tablet comprising a core region with one or more immediate release layers or portions containing a proton pump inhibitor and one or more extended release layers or portions of the proton pump inhibitor, where the ratio of proton pump inhibitor in the one or more immediate release layers or portions to that in one or more extended release layers or portions ranges from 10:90 to 90:10, with the proviso that the proton pump inhibitor does not comprise Omeprazole or a salt thereof.

3. The multilayer tablet of claim 2 wherein the proton pump inhibitor is Esomeprazole, Lansoprazole, Pantoprazole or Rabeprazole sodium or a salt thereof.

4. The multilayer tablet of claim 1 or 2 further comprising an enteric coating layer or extended release coating layer over said core region.

5. The multilayer tablet of claim 4 further comprising a subcoating on said core region between said core region and said enteric coating layer or said extended release coating layer.

6. The multilayer tablet of claims 1 or 2 wherein the proton pump inhibitor is released at a rate which achieves an acceptable plasma Cₘₐₓ and AUC as compared to capsules and/or tablets consisting of multiple unit pellets of the proton pump inhibitor.

7. A method for producing the multilayer tablet of claim 1 or 2 comprising compressing one or more immediate release layers or portion of the proton pump inhibitor and one or more extended release layers or portions of the proton pump inhibitor together into a single core region of multilayer tablet and coating the core region with an enteric polymer or an extended release polymer.

8. The method of claim 7 further comprising applying a subcoating to the core region prior to applying the enteric polymer coating or extended release coating.

9. A multilayer tablet comprising a core region containing a proton pump inhibitor, a polymer layer coating said core region which provides for controlled release of the proton pump inhibitor from said core region, and a proton pump inhibitor containing layer coating said polymer layer which rapidly releases the proton pump inhibitor on contact with fluid.

10. A multilayer tablet comprising a core region containing a proton pump inhibitor, a polymer layer coating said core region which provides for controlled release of the proton pump inhibitor from said core region, and a proton pump inhibitor containing layer coating said polymer layer which rapidly releases the proton pump inhibitor on contact with fluid, with the proviso that the proton pump inhibitor does not comprise Omeprazole or a salt thereof.

11. The multilayer tablet of claim 10 wherein the proton pump inhibitor is Esomeprazole, Lansoprazole, Pantoprazole or Rabeprazole sodium or a salt thereof.

12. The multilayer tablet of claim 9 or 10 further comprising an enteric coating layer or extended release coating layer over said core region.

13. The multilayer tablet of claim 12 further comprising a subcoating on said core region between said core region and said enteric coating or extended release coating.

14. The multilayer tablet of claim 12 further comprising a subcoating on said polymer layer between said polymer layer and enteric coating or extended release coating or said proton pump inhibitor containing layer.

15. The multilayer tablet of claims 9 or 10 wherein the proton pump inhibitor is released at a rate which achieves acceptable plasma Cₘₐₓ and AUC as compared to capsules and/or tablets consisting of multiple unit pellets of the proton pump inhibitor.

16. A method for treating dyspepsia, peptic ulcer disease, gastroesophageal reflux disease and Zollinger-Ellison syndrome which comprises administering to a patient the multilayer tablet of claims 1, 2, 3, 9, 10 or 11.
